(19) United States
(12) Patent Application Publication
(10) Pub. No.: US 2009/0192579 A1
(43) Pub. Date: Jul. 30, 2009

(54) IMPLANTATION METHODS, SYSTEMS AND TOOLS FOR INTRAVASCULAR IMPLANTABLE DEVICES

(76) Inventors: Terrance Ransbury, Chapel Hill, NC (US); Kevin Holbrook, Chapel Hill, NC (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/327,808

(22) Filed: Dec. 3, 2008

Related U.S. Application Data
(60) Provisional application No. 61/005,354, filed on Dec. 3, 2007.

(51) Int. Cl. A61N 1/05

(52) U.S. Cl. 607/119

(57) ABSTRACT

Methods, systems and tools for implanting long-term active therapeutic medical devices referred to as intravascular implantable devices (IID) within a patient's vasculature are disclosed. In one embodiment, the implantation techniques and instruments of the present invention are adapted for implanting an IID having only a single anchoring arrangement positioned proximate a distal portion of the device for anchoring within a vessel located superior to the heart, i.e. above the heart in a direction toward the head of a patient. Other embodiments of the invention include various methods and tools for delivering and implanting an IID, delivering and securing an anchor, and delivering and implanting one or more leads.

### RELATED APPLICATIONS

The present invention claims priority to U.S. Provisional Patent Application No. 61/005,354, entitled “Implantation Methods, Systems and Tools for Intravascular Implantable Devices,” filed Dec. 3, 2007 which is hereby incorporated by reference in its entirety.

### FIELD OF THE INVENTION

The present invention generally relates to devices, systems and methods for diagnosing and treating patients. In particular, the invention relates to methods, systems and tools for implanting long-term active therapeutic medical devices into the vasculature of a patient.

### BACKGROUND OF THE INVENTION

Implantable devices that provide long-term active therapies such as pacemakers, defibrillators, and implantable cardioverter defibrillators (“ICDs”) have been successfully implanted in patients for years for treatment of heart rhythm conditions. Pacemakers are implanted to detect periods of bradycardia and deliver low energy electrical stimuli to increase the heart rate. ICDs are implanted in patients to cardiovert or defibrillate the heart by delivering high energy electrical stimuli to slow or reset the heart rate in the event a ventricular tachycardia (VT) or ventricular fibrillation (VF) is detected. Another type of implantable device detects an atrial fibrillation (AF) episode and delivers an electrical stimuli to the atria to restore electrical coordination between the upper and lower chambers of the heart. The current generation for all of these implantable cardiac rhythm management (CRM) devices are typically can-shaped devices implanted under the skin that deliver electrical stimuli via leads that implanted in the heart via the patient's vascular system.

Conventional pacemakers and ICDs are implanted subcutaneously, typically in the pectoral region. Conventional implantable pulse generators such as pacemakers and ICDs use conventional leads in the form of elongated, floppy lead bodies that insulate, seal and protect one or more conductors which transmit electrical pulses between the pulse generator and one or more electrodes on the lead. The one or more intravascular leads associated with a conventional pacemaker device or ICD are typically not integrated with the device; instead, a header is provided on the device for connecting the one or more leads to the device. The lead tip is affixed in, on, or near the heart, depending on the desired treatment.

To implant the one or more intravascular leads for a conventional device implanted subcutaneously in the pectoral region, the lead is passed into the subclavian vein, routed through the superior vena cava, and down into the heart. Most intravascular cardiac leads for conventional CRM devices are guided with use of a stylet that is inserted into a lumen within the lead body accessed via the proximal end of the lead, with the stylet used to direct the distal end of the lead into the desired position. The lead tip is affixed in, on, or near the heart, depending on the desired treatment.

Once in position, the distal end of the lead may be fixed in position within the heart, either by passive fixation or active fixation. Passive fixation leads may feature protruding tines, and/or a steroid-coated lead tip, such that when the lead tip is inserted to the desired location, biological processes in the heart tissue will secure the lead in place. Active fixation leads typically include a helix or corkscrew tip, and this tip is secured directly into the myocardium. Active fixation offers more precise placement of the lead, as well as greater stability when secured in the heart.

The one or more leads associated with a conventional CRM device are typically not integrated with the device; instead, a header is provided on the device for connecting the one or more leads to the device. Such a connection arrangement between the conventional device and the lead allows for access to the lumen within the lead via the proximal end. Implantation of the device typically follows implantation of the lead. The lead is connected to the device, and the device is then secured in the patient.

While stylet-based delivery of cardiac leads is the most prevalent technique used, other techniques for cardiac lead delivery and fixation have also been developed. One such technique is an over-the-wire technique in which the lead is advanced over a guide wire. Different versions of this over-the-wire technique are described, for example, in U.S. Pat. Nos. 5,003,990, 5,304,218 and 6,129,749. Another technique involves the use of a guide catheter as a pusher for delivering the lead into position within the heart. Different versions of a guide catheter technique are described in U.S. Pat. Nos. 5,571,161, 6,185,464, 7,018,384 and 7,092,765.

Next generation long-term active implantable devices may take the form of elongated intravascular devices that are implanted within the patient's vascular system, instead of under the skin. Examples of these intravascular implantable devices (IIDs) are described, for example, in U.S. Pat. No. 7,082,336 and U.S. Published Patent Application Nos. 2005/0043765A1, 2005/0228471A1 and 2006/0217779A1. These devices contain electric circuitry and/or electronic components that must be hermetically sealed to prevent damage to the electronic components and the release of contaminants into the bloodstream. Due to the length of these implantable devices, which in some cases can be approximately 10-60 cm in length, the devices must be flexible enough to move through the vasculature while being sufficiently rigid to protect the internal components.

As described in U.S. Pat. No. 7,082,336, techniques for implanting such an intravascular implantable device generally begin by obtaining access to the vasculature of the patient through a puncture made in a vessel, such as one of the femoral veins in the leg. In some embodiments described in U.S. Pat. No. 7,082,336, an over-the-wire implant technique was used for both the lead and the elongated device, utilizing a pusher in the form of a mandrel that was detachably coupled to the proximal end of the elongated device to push the device into a position where the device could be anchored using an anchoring system. While such a pushing arrangement permits positive control of the proximal end of the device, the mechanical nature of the mandrel coupling can complicate the construction and implantation of the device and can present challenges with respect to issues of effective hermetic sealing and potential thrombosis formation near this region of the device, as well as complicating lead placement on the proximal end of the device. In addition, next-generation IID's may include a lead integrally connected to the proximal end of the IID, preventing the use of a mandrel pusher.

As described in some of the embodiments shown in U.S. Pat. No. 7,082,336 and U.S. Published Patent Application No. 2004/0249431, the anchoring system was arranged proximate the middle of the intravascular implantable device so as to be positioned in the vena cava within the thorax. This arrangement anchored the intravascular implantable device near the middle of the torso of the patient, at a location generally corresponding to the diaphragm. In some embodiments, the anchoring system was integrated with the body of the intravascular implantable device. In other embodiments, the anchoring system was a separate device that was used to pin the body of the intravascular implantable device in position between the stent and the vessel wall.

While the implantation techniques described in the above-referenced patents and published applications were adequate for the location and deployment of the anchoring system near the middle of the patient's torso, the location and deployment of an anchoring system at other locations in the vasculature can present additional challenges. Improvements to intravascular implantable devices provide for an IID that may be anchored superior to the heart, such as in the subclavian vein. Methods of anchoring IID's superior to the heart, as well as descriptions of devices suitable for anchoring in such a manner, are described in U.S. Published Patent Application Nos. 2008/0167702 and 2008/0147168.

In addition to challenges associated with anchoring a device superior to the heart, due to the many differences between conventional implantable CRM devices and intravascular implantable devices, well-known methods and devices for traditional lead introduction and fixation are not necessarily applicable to next-generation IIDs. For example, intravascular lead(s) for conventional CRM devices are usually introduced into the heart by way of the superior vena cava, while for intravascular implantable devices, the lead(s) are usually introduced to the heart via the inferior vena cava. The maneuvering of the lead from the inferior vena cava into the right atrium and on into the right ventricle is especially problematic using prior lead delivery systems and methods. Additionally, the cardiac lead of an IID is unlike a cardiac lead for a conventional CRM device in that the proximal end of the lead is generally unavailable for access to aid in the implantation of the lead, such as by use of a steerable stylet introduced into a lumen of the lead through the proximal end of the lead.

Previous approaches for delivering cardiac leads into the heart for intravascular implantable devices are disclosed in U.S. Pat. No. 7,082,336. In one approach, the lead includes a cuff, through which a guidewire is introduced through a distal end of the lead while the lead is outside of the body and the device is already implanted. The guidewire is steered to the fixation site, and a pusher is introduced onto the free end of the wire. The pusher is advanced against the lead cuff, and the lead is pushed along the guidewire to the fixation location. A fixation element is provided on the lead tip for securing the lead at its desired location.

A further challenge associated with implantation of IIDs is maintaining the incision used for insertion of the device, anchor, and lead. Conventional intravascular catheters for use with temporary procedures like angiograms or for implanting long-term passive devices like stents typically have diameters of less than 15 French (<5 mm) and mostly less than about 12 French (<4 mm). However, diameters for IID's are typically greater than 15-18 French (5-6 mm) and potentially up to 25 French (<9 mm). Therefore, the size of the vessel puncture, the manner in which blood is retained, and instruments that can access the vasculature through the vessel puncture all present problems that can be much different than vascular access techniques for devices smaller than about 15 French.

While intravascular implantable devices represent a significant improvement over conventional long-term active implantable devices that are implanted subcutaneously, there are opportunities to improve and refine the implantation techniques, system and tools for implanting such intravascular devices. It would be desirable to provide improved methods, systems and tools for implanting such intravascular implantable devices that can simplify the implantation of these devices so as improve the effectiveness and ease of the procedure.

### SUMMARY OF THE INVENTION

The present invention is directed to methods, systems and tools for implanting long-term active therapeutic medical devices referred to as an intravascular implantable device (IID) within a patient's vasculature. Implantation of an IID generally includes maintaining a vessel puncture open during the procedure, delivery and fixation of the device, delivery and fixation of one or more anchors to retain the device within the vasculature, and delivery and fixation of one or more leads, with these procedures not necessarily carried out in this order. In one embodiment, the implantation techniques and instruments of the present invention are adapted for implanting an IID having only a single anchoring arrangement positioned proximate a distal portion of the device for anchoring within a vessel located superior to the heart, i.e. above the heart in a direction toward the head of a patient.

Referring now to implantation of the device, in one embodiment improved methods and apparatuses for positioning an intravascular implantable device (IID) in a patient's vasculature utilize a device delivery system having an elongated flexible body. The device delivery system can have an elongated flexible body adapted to be temporarily implanted into a patient's vasculature. The body can have a proximal end and a distal end and can include a flexible wire. A handle can be operably connected to the proximal end of the device body and a grasper mechanism can be connected to the distal end of the flexible wire. The grasper mechanism can be configured to releasably grasp an IID by closing a releasable honda that is selectively controllable with the handle around the IID. In one embodiment, the releasable honda can be closed around the IID by inserting an end portion of the releasable honda into a collar section located at the distal end of the device body. In another embodiment, the releasable honda can be closed around the IID by connecting a stylet projecting from device body with the releasable honda. The releasable honda can be configured to release its grasp on the IID via operation of the handle.

In one embodiment of positioning an IID in a patient's body, the grasper device delivery system is used to grasp the IID by closing a releasable honda around the IID. The IID can then be guided through the introducer catheter and through the patient's vasculature by controlling the device delivery system. Once the IID is in a desired location, the handle of the device delivery system can be used to release the IID. In one embodiment, at least a portion of the device delivery system can then be withdrawn from the patient's vasculature. In another embodiment, device delivery system can retain position control until after a lead associated with the IID is implanted.

In a further embodiment, the device delivery system is used in conjunction with a guide wire such that IID delivery is accomplished with an over-the-wire technique. A standard Seldinger technique is performed, wherein an incision is formed in the femoral vein and an introducer is inserted into the incision to keep the vein open during the procedure. A guide catheter is inserted through the introducer and a guide wire is directed into a vessel superior to the heart. The guide catheter is removed, leaving the guide wire in place. In one embodiment, the distal portion or tip of the device includes a passage for the wire, while in another embodiment, the device body includes a passage for the wire. The device is then inserted onto the guide wire, and the device is manually inserted into the vasculature until the proximal end of the device reaches the introducer. A delivery device catheter is then used to maneuver the device to its desired location. The device delivery catheter is adapted to grasp the IID. Grasping may be achieved through the use of a mechanism such as a retractable or releasable lasso. In one embodiment, the device delivery catheter is configured to grasp the proximal end of the device, such that the IID is pushed to its desired location. The device delivery system provides positive control of the proximal end of the IID during delivery, while positive control of the distal end of the IID is provided by the distal end or tip of the IID passing along the guidewire.

Referring now to anchor delivery and deployment, in one embodiment anchor delivery is accomplished with an anchor delivery catheter. The anchor delivery catheter is configured to deliver an anchor, such as an intravascular stent, to a desired location and then deploy the anchor. In one embodiment, the anchor delivery catheter is inserted through the introducer, and steered through the vasculature to the desired location. The anchor is preferably delivered in a compressed configuration, and expands when deployed from the anchor delivery catheter.

In another embodiment, the anchor is delivered via an over-the-wire technique. The anchor delivery catheter may be adapted to communicate with a guide wire, such that a guide wire is introduced into the vasculature, and the anchor is directed along the guide wire to a desired location where it is deployed. In a further embodiment, over-the-wire anchor delivery may be accomplished via the same wire that is used for device delivery. Subsequent to device delivery, the anchor is directed along the same guidewire until reaching the location where the guidewire enters the IID. At this point the guidewire is retracted from the IID and advanced through the vasculature past the IID. The anchor is guided along this path until reaching the desired deployment location, such as alongside the IID tip or tether.

Referring now to delivery and fixation of the one or more leads, in one embodiment the IID is provided with a single lead integrally connected to the proximal end of the device body. Delivery and implantation of the lead is typically performed subsequent to the device delivery. To deliver a lead from the inferior vena cava to the interior of the heart, such as the right atrium or right ventricle, the lead must be maneuvered through the acute angle between the inferior vena cava and the heart. Lead delivery devices may include the ability to articulate and/or extend, facilitating the delivery of the lead into the heart apex. Suitable lead delivery devices provide positive control of the lead, or at least the distal end of the lead, during implantation.

In one embodiment, the grasper-style lead delivery tool is of identical construction and function to the device delivery tool described above and is configured to releasably grasp a portion of the lead body. The lead delivery tool includes the ability to articulate, rotate and/or extend to facilitate delivery of the lead to a desired location.

Referring now to maintaining access to a vessel during the procedure, in one embodiment the method includes the utilization of an introducer catheter with a multi-stage large bore hemostasis valve. The introducer catheter includes a proximal end, a distal end, and an access lumen extending there between. A tube coupled to the distal end may also be included, the tube being configured to extend into the vasculature of the patient during the procedure. The multi-stage large bore hemostasis valve is disposed at the proximal end, which remains outside the patient's body during the procedure. The multi-stage large bore hemostasis valve may be a multi stage device with a large diameter bore of greater than 25 French. A radially expanding first stage with an iris effect overlaps the second stage. The second stage serves as a retardant to limit fluid flow during open operation of the first stage. The introducer sheath hub may include at least one hard stop to prevent the cap from backing out completely from the body when rotated.

A compressible silicone gland may be used to achieve hemostasis around a broad range of devices ranging from 1-26 French and to close substantially this broad range of openings when no devices are inserted. A second stop prevents or limits the over compression of the gland.

The above summary of the various embodiments of the invention is not intended to describe each illustrated embodiment or every implementation of the invention. This summary represents a simplified overview of certain aspects of the invention to facilitate a basic understanding of the invention and is not intended to identify key or critical elements of the invention or delineate the scope of the invention.

### BRIEF DESCRIPTION OF THE DRAWINGS

The invention may be more completely understood in consideration of the following detailed description of various embodiments of the invention in connection with the accompanying drawings, in which:

FIG. 1 is a perspective illustration depicting human cardiac anatomy.

FIG. 2 is a schematic generally depicting components of an intravascular electrophysiological system according to one aspect of the present invention.

FIG. 3 is a schematic of one embodiment of an intravascular implantable device according to the present invention.

FIG. 4A is a side view of an introducer sheath according to one aspect of the present invention.

FIG. 4B is a perspective exploded view of the introducer sheath of FIG. 5A.

FIG. 5A is a perspective view of a hemostasis valve for use with an introducer sheath according to one aspect of the present invention.

FIG. 5B is an exploded view of the hemostasis valve of FIG. 6A.

FIG. 6A is a perspective view of a delivery system according to one aspect of the present invention.

FIG. 6B is an exploded perspective view of the delivery system of FIG. 6A.

FIG. 6C is a top plan view of the delivery system of FIG. 6A.

FIG. 6D is a side plan view of the delivery system of FIG. 6A.

FIG. 6E is an end plan view of the delivery system of FIG. 6A.

FIG. 7A is a perspective view of a device body of a delivery system according to one aspect of the present invention.

FIG. 7B is a top plan view of the device body of FIG. 7A, depicted without a sheath for clarity.

FIG. 7C is a side plan view of the device body of FIG. 7B.

FIG. 7D is a top plan view of the device body of FIG. 7A.

FIG. 7E is a side plan view of the device body of FIG. 7A.

FIG. 8A is a perspective view of a grasper mechanism according to one aspect of the present invention in a free state.

FIG. 8B is a perspective view of the grasper mechanism of FIG. 7A in an engaged state.

FIG. 9A is a side plan view of a delivery system grasping an intravascular implantable device according to one aspect of the present invention.

FIG. 9B is a perspective view of the delivery system grasping an intravascular implantable device of FIG. 8A.

FIG. 10 is a view of an intravascular implantable device being guided into a patient's body with a delivery system according to one aspect of the present invention.

FIG. 11 is a perspective view of a deployed anchor according to one aspect of the present invention.

FIG. 12A is a perspective view of an anchor delivery catheter according to one aspect of the present invention.

FIG. 12B is a perspective exploded view of the anchor delivery catheter of FIG. 12A.

FIG. 12C is a partial detail of the anchor delivery catheter of FIG. 12B.

FIG. 12D is a partial detail of the anchor delivery catheter of FIG. 12B.

FIG. 13A is a view of an intravascular implantable electrophysiology device being guided through the inferior vena cava of a patient according to one aspect of the present invention.

FIG. 13B is a view of the intravascular implantable electrophysiology device of FIG. 13A positioned fully within the vasculature of the patient, with a cardiac lead extending from the proximal end of the device and being grasped by a lead delivery system according to one aspect of the present invention.

FIG. 13C is a view of a cardiac lead being guided through the vasculature with a lead delivery system according to one aspect of the present invention.

FIG. 13D is a view of a cardiac lead being guided to a desired location within the heart of a patient by a lead delivery system according to one aspect of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE DRAWINGS

In the following detailed description of the present invention, numerous specific details are set forth in order to provide a thorough understanding of the present invention. However, one skilled in the art will recognize that the present invention may be practiced without these specific details. In other instances, well-known methods, procedures, and components have not been described in detail so as to not unnecessarily obscure aspects of the present invention.

The present disclosure describes intravascular electrophysiological systems that may be used for a variety of functions. These functions include defibrillation, pacing and/or cardioversion. In general, the elements of the systems described herein include at least one device body and typically, but optionally, at least one lead coupled to the body. One or more retention devices may facilitate the retension of the device body and/or leads or other elements within the vasculature. Also described are components such as catheters, tools, stylets and/or guide wires used to facilitate implantation of the system.

### Anatomy

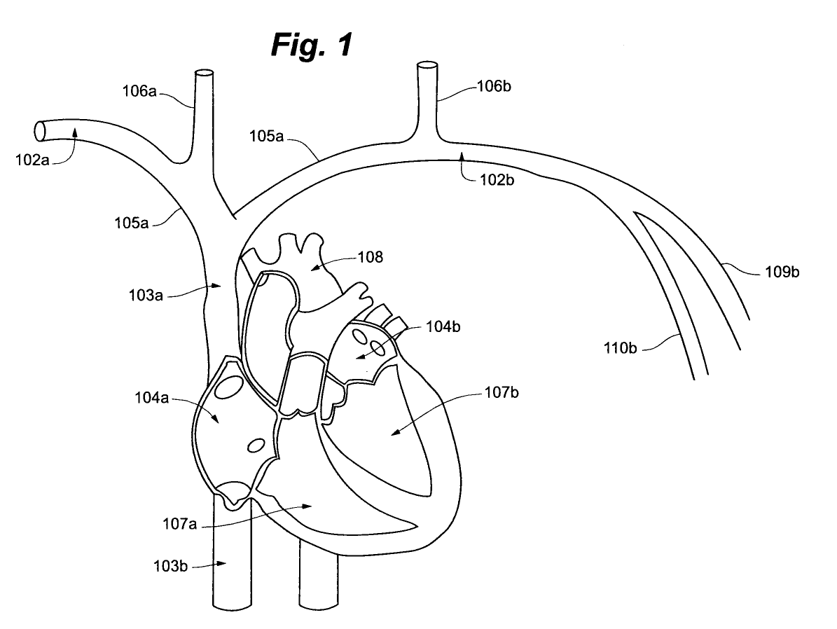

Referring to FIG. 1, the general cardiac anatomy of a human is depicted, including the heart and major vessels. The following anatomic locations are shown and identified by the listed reference numerals: Right Subclavian 102a, Left Subclavian 102b, Superior Vena Cava (SVC) 103a, Inferior Vena Cava (IVC) 103b, Right Atrium (RA) 104a, Left Atrium (LA) 104b, Right Innominate/Brachiocephalic Vein 105a, Left Innominate/Brachiocephalic Vein 105b, Right Internal Jugular Vein 106a, Left Internal Jugular Vein 106b, Right Ventricle (RV) 107a, Left Ventricle (LV) 107b, Aortic Arch 108, Descending Aorta 109, Right Cephalic Vein 109a (not shown in FIG. 1), Left Cephalic Vein 109b, Right Axillary Vein 110a (not shown in FIG. 1) and Left Axillary Vein 110b. Reference number 100 refers generally to vessels and/or vessel walls within the human body.

### The Kit

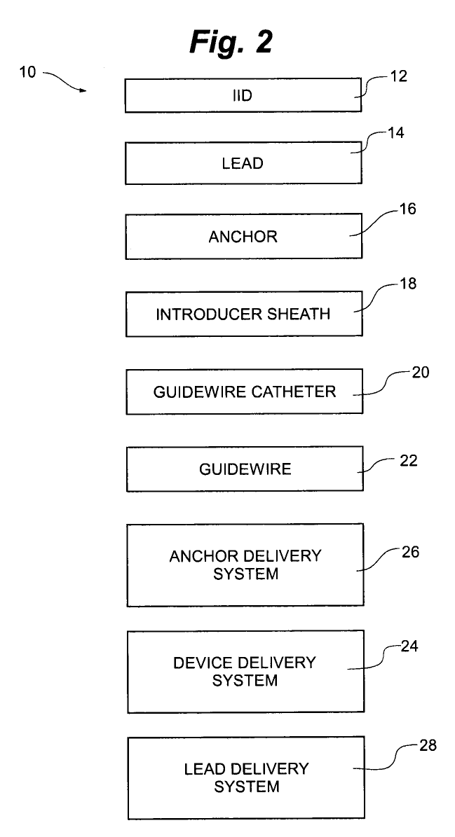

One configuration of the components of an electrophysiological treatment system 10 is depicted in FIG. 2. System 10 generally includes an intravascular implantable device (IID) 12 having a lead 14, the device being retained within a vessel 100 by an anchor 16. An introducer sheath 18 is provided for implantation of system 10. A guidewire catheter 20 is provided to deploy a guidewire 22 within the vasculature of a patient. A device delivery system 24 may optionally be used in conjunction with guidewire 22 to navigate the IID to the desired location. An anchor delivery system 26 may also rely on guidewire 22 to deliver anchor 16 to the desired location. Anchor delivery system 26 may also include a means for fixing or deploying anchor 16. A lead delivery system 28 is provided for maneuvering lead 14 to its desired location.

In one embodiment, instructions for implanting the system 10 in accordance with the various embodiments described herein in the form of printed or electronically, optically or magnetically stored information to be displayed, for example, are provided as part of a kit or assemblage of items prior to surgical implantation of the system 10. In another embodiment, instructions for implanting the system 10 in accordance with the various embodiments described herein are provided, for example, by a manufacturer or supplier of system 10, separately from providing the system 10, such as by way of information that is accessible using the Internet or by way of seminars, lectures, training sessions or the like.

In one embodiment, implantation of system 10 is done using an over-the-wire technique, which generally proceeds as follows. An incision is formed in the femoral vein and an introducer is inserted into the incision to keep the vein open during the procedure, the introducer being configured to allow insertion of devices into the vasculature while preventing the loss of blood. A guide catheter is inserted through the introducer and a guide wire is directed into a vessel superior to the heart near the target anchor location. Fluoroscopy is preferably utilized to aid in all aspects of implantation. The guide catheter is removed, leaving the guide wire in place. A choice is then made as to what is implanted next: the anchor, the device, or the lead. The order of implantation of these components will depend on the desired application. For purposes of illustration, an embodiment wherein the device is delivered first, then the anchor, then the lead, will be discussed.

A distal portion of the IID containing a guidewire passage is inserted over the guide wire, and the IID is manually inserted through the introducer sheath as far as possible. A dissolvable, lubricious coating may be applied to the IID to aid in delivery of the device through the vasculature. A device delivery system then grasps the proximal end of the IID, and the IID is guided through the vasculature to the desired location superior of the heart. In one embodiment, the device delivery system is then removed from the vasculature.

An anchor delivery system is then directed along the guide wire to a position proximate the distal portion of the IID. The guide wire is retracted from the distal portion of the IID and re-advanced alongside the tip portion of the IID. An anchor can be inserted onto the anchoring delivery system and introduced advanced along the guide wire to a position proximate the distal tip portion of the IID. Once in a desired position, the anchor may be deployed and the guide wire and anchor delivery system can then removed. In one embodiment, the anchor is of the passive type, such as holding the IID between the anchor and the vessel wall. In another embodiment, the anchor may be of an active type, requiring manipulation of the IID to facilitate a connection with the deployed anchor. In such an embodiment, the DDC may be used to complete the anchoring step.

The lead may then be introduced into the heart with a lead delivery catheter. In one embodiment, the lead body is grasped and the lead is pushed through the inferior vena cava up into the superior vena cava, going past the heart. The lead is then manipulated into the heart. In another embodiment, the lead is advanced through the inferior vena cava and directed into the heart. Use of articulation and/or extension allows the lead to be positioned in the desired location, without damaging or interfering with tissues in the heart. The lead delivery catheter is then released and removed. After the device has been delivered and anchored, and the lead has been implanted, the introducer sheath may be removed and the femoral incision closed.

### Structure of the Intravascular Implantable Device

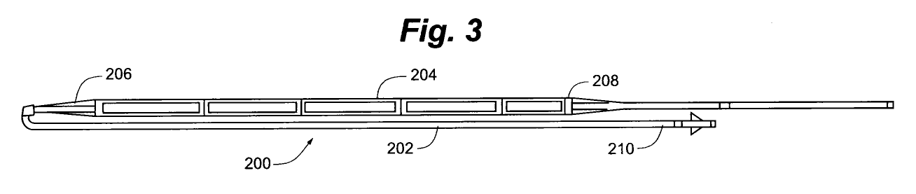

Referring generally to FIG. 3, an IID 200 according to one aspect of the present invention is depicted. In one embodiment, the IID 200 includes components known in the art to be necessary to carry out the system functions. For example, the IID 200 may include one or more pulse generators, including associated batteries, capacitors, microprocessors, and circuitry for generating electrophysiological pulses for defibrillation, cardioversion and/or pacing. The IID 200 also includes detection circuitry for detecting arrhythmias or other abnormal activity of the heart. The specific components to be provided in the device will depend upon the application for the device, and specifically whether the device is intended to perform defibrillation, cardioversion and/or pacing along with its sensing functions.

The IID 200 is proportioned to be passed into the vasculature and to be anchored within the patient's vasculature with minimal obstruction to blood flow. Suitable sites for the IID 200 may include, but are not limited to, the venous system using access through the right or left femoral vein or the subclavian or brachiocephalic veins, or the arterial system using access through one of the femoral arteries. Thus, the housing of the IID 200 preferably has a streamlined maximum cross sectional diameter which may be in the range of 3-15 mm or less, with a most preferred maximum cross-sectional diameter of 3-8 mm or less. The cross-sectional area of the device 200 in the transverse direction (i.e. transecting the longitudinal axis) should be as small as possible while still accommodating the required components. The cross-section of the device 200 (transecting the longitudinal axis) may have a circular cross-section, although other cross-sections including crescent, flattened, or elliptical cross-sections may also be used. It can be desirable to provide the device with a smooth continuous contour so as to avoid voids or recesses that could encourage thrombus formation on the device.

The lead 202 may be integrated with the device body 204, or attachable to the device body 204 in situ or prior to implantation, or the lead 202 may be integral with the device as an extension of the device itself. More than one lead 202 may be provided. Leads 202 may be included on the proximal end 206 of the device body 204, on the distal end 208 of the device body 204, generally on the device body, and/or any combination thereof. A lead 202 includes one or more defibrillation and/or pacing electrodes and may also be equipped to sense electrical activity of the heart. Monitoring of the heart's electrical activity can be needed to detect the onset of an arrhythmia. Activity sensed by the sensing electrode(s) is used by the device electronics to trigger delivery of a defibrillation shock. The lead 202 may be a conventional defibrillation/pacing lead, although alternative lead configurations may be desirable if warranted by the desired placement of the IID 200 and lead 202 within the body.

For leads 202 that are to be positioned within a chamber of the heart, the leads 202 may be the helical screw-in or tined variety for fixation to the cardiac tissue, and/or they may have steroid-eluding tips to facilitate tissue in-growth for fixation purposes. If a detachable tip is used, the lead tip 210 may be left within the chamber of the heart when the remainder of the lead 202 is removed, so as to prevent damage to the heart tissue as could occur upon extraction of the tined tip.

The leads 202 may include non-thrombogenic and/or non-proliferative surfaces or coatings, for example, the leads 202 may include a coating that is anti-thrombogenic (e.g. perfluorocarbon coatings applied using supercritical carbon dioxide) so as to prevent thrombus formation on the lead 202. It is also beneficial for the coating to have anti-proliferative properties so as to minimize endothelialization or cellular ingrowth, since minimizing growth into or onto the lead 202 will help minimize vascular trauma when the device is explanted. The coating may thus also be one which elutes anti-thrombogenic compositions (e.g. heparin sulfate) and/or compositions that inhibit cellular in-growth and/or immunosuppressive agents.

Thus, it should be appreciated that in this disclosure the term “lead” is used to mean an element that includes conductors and electrodes and that thus may be positioned somewhat remotely from the circuitry that energizes the electrodes. In other embodiments, leads may include elements that are simply extensions or tapers of the IID itself (such as the portion of the device at which electrodes are located) as well as more conventional intravascular leads.

In accordance with one embodiment of the present invention, the IID 200 can include at a distal end 208 an anchor attachment feature that allows the IID 200 to be disposed within the vasculature. An anchor detachment feature may be included so as to allow for removal of the IID 200 at a later date without damaging the vasculature by removing the anchor. An anchor zone may be disposed between the anchor attachment feature and the detachment feature for positioning the IID 200 between an anchor and the vasculature wall. In one embodiment, a telemetry antenna may be disposed axially along the distal end 208 proximate the anchor zone.

IID 200 may also be provided with a lumen for passage of a guidewire therethrough, such as described in U.S. Published Application Nos. 2008/0147168 and 2008/0167702, the disclosures of which are hereby incorporated by reference. The lumen may be included in distal portion 208 of the device body 204. In another embodiment, IID 200 includes a tip portion coupled to distal portion 208 of device body 204. Tip portion may include an internal telemetry antenna, a guidewire lumen extending the length of the tip, and tip portion may further provide an anchor attachment feature.

Additional disclosure pertaining to the structure and layout of intravascular implantable devices, as well as leads and anchors, can be found in U.S. Published Patent Application Nos. 2006/0217779, 2007/0265673, 2008/0147168, and 2008/0167702, the disclosures of which are hereby incorporated by reference in their entireties.

### Introducer Sheath

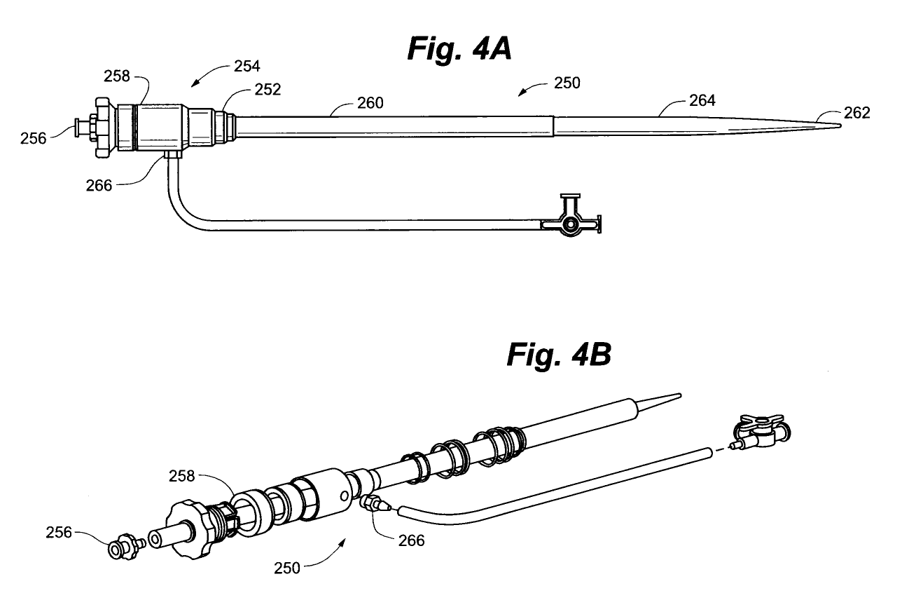

To facilitate introduction of the IID, an introducer 250 such as depicted in FIGS. 4A-4B may be utilized as a mechanism to access the vascular system of the patient. In one embodiment, the sheath introducer 250 includes at a proximal end 252 a removable cap assembly 254 with an iris or box fold valve 256 followed upstream by a rotating hemostasis valve 258 that extends into the introducer shaft 260. A tapered nose cone dilator tip 262 is disposed at the distal end 264 of the introducer 250. The introducer 250 defines a central aperture extending along the longitudinal axis. A flush port 266 can be operably disposed upstream of the hemostasis valve 258 so as to be in fluid communication with the central aperture.

The laminated introducer shaft 260 of one embodiment of the introducer 250 may include a coil embedded within the shaft. The shaft 260 may include a coating or be constructed of one or more lubricous materials so as to present a slippery inner surface for the insertion and withdrawal of various medical apparatus as described herein. The coil can be a circular wire placed in compression liner. The spacing between the coils is predetermined so that the pitch is optimized for strength and flexibility. In a first embodiment, the coil diameter is 0.007 inches with a pitch of 0.031 inches. A laminated liner is positioned about the shaft 260. The laminated liner includes a durometer of between 50-60, and in one embodiment about 55, on the Shore D hardness scale. In one embodiment, the liner overlaps the ends of shaft 260.

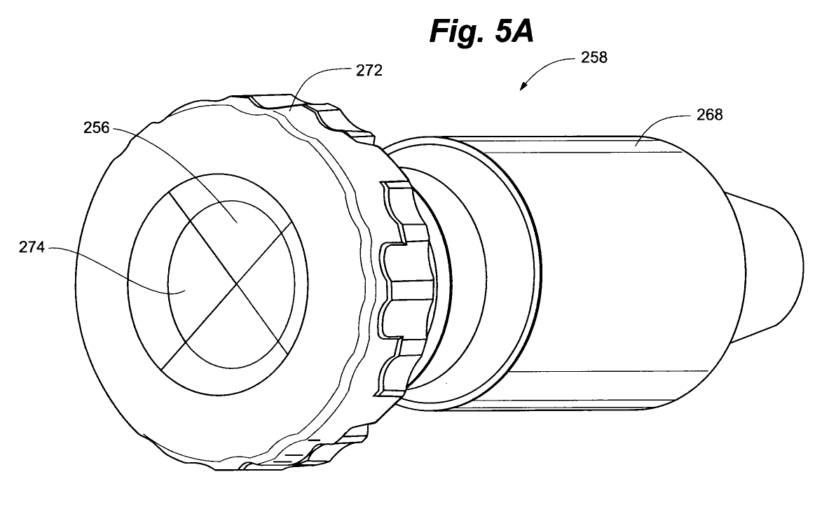

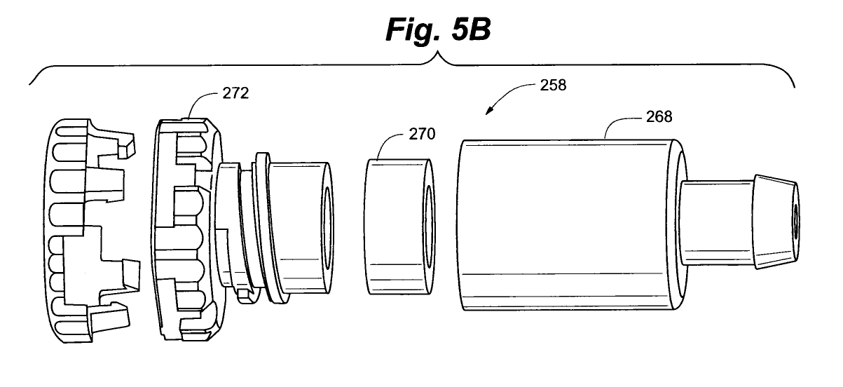

One embodiment of the hemostasis valve 258 depicted in FIGS. 5A-5B may include a valve body 268, a seal 270, a second stage valve 256 having an iris or box valve configuration and a screw cap 272 all controlling flow through the central aperture. The screw cap 272 includes a plurality of clips to slidingly lock onto the second stage valve through a clip flange. The seal 270 may be a compressible silicon gland that is disposed within a valve body recess. The second stage valve 256 can include at a first end multiple petals 274 or leaflets that mate in a box-like fashion. The clips help to compress the leaflets 274 on to the second stage valve 256. The opposing end of second stage valve 256 can include a threaded section that mates within body 268. The body 268 can include a hard stop to prevent the complete removal of second stage valve 256 and a second hard stop to prevent the second stage valve 256 from over compression of seal 270.

Additional disclosure relating to introducers that may be used with the methods and systems of the present invention can be found in U.S. Pat. No. 5,921,698 to Lampropoulos et al., U.S. Pat. No. 6,458,103 to Albert et al., and U.S. Pat. No. 6,572,590 to Stevens et al., the disclosures of each of which are incorporated by reference in their entireties.

### Device Delivery

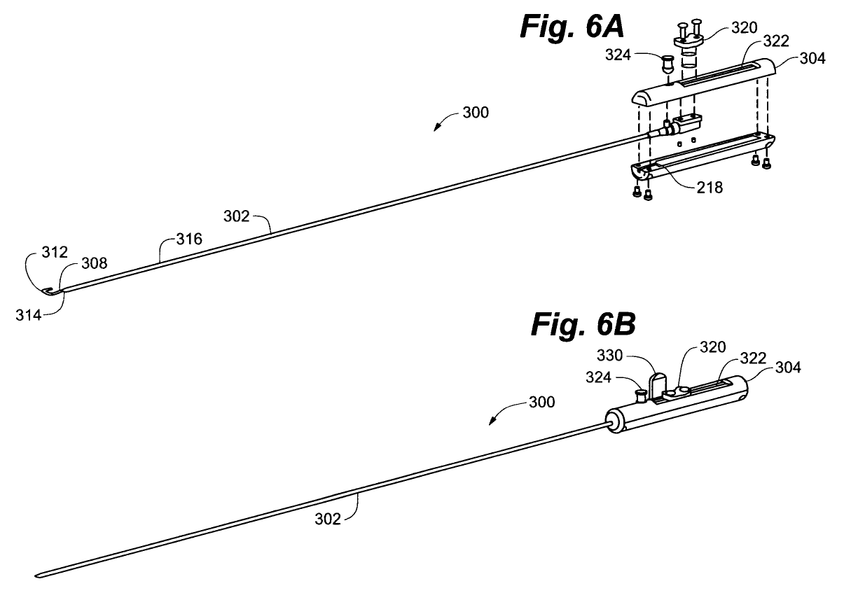

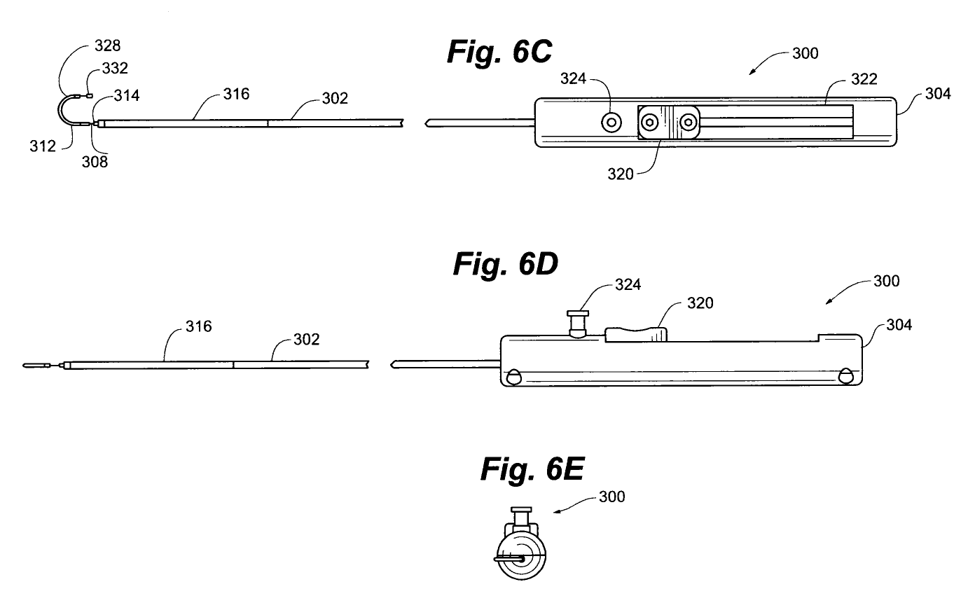

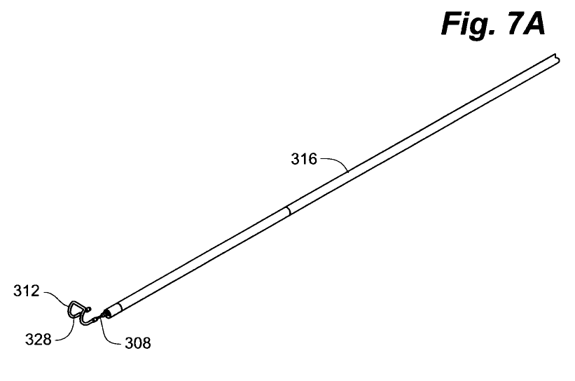

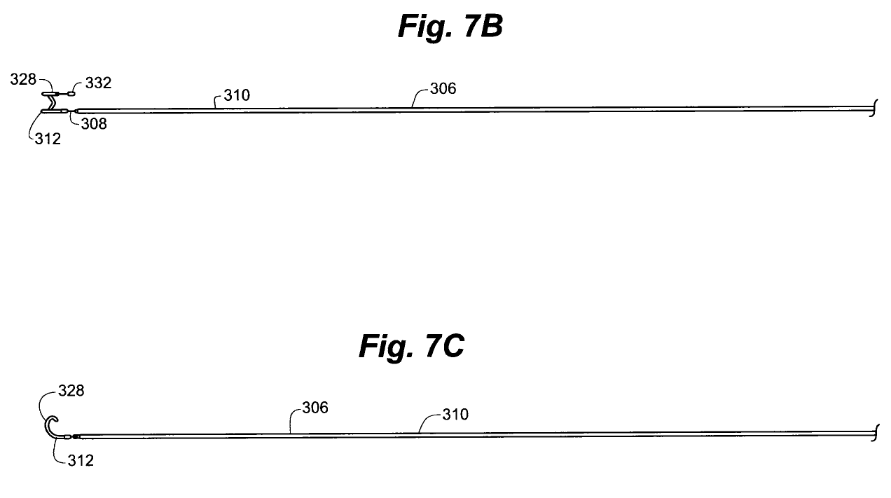

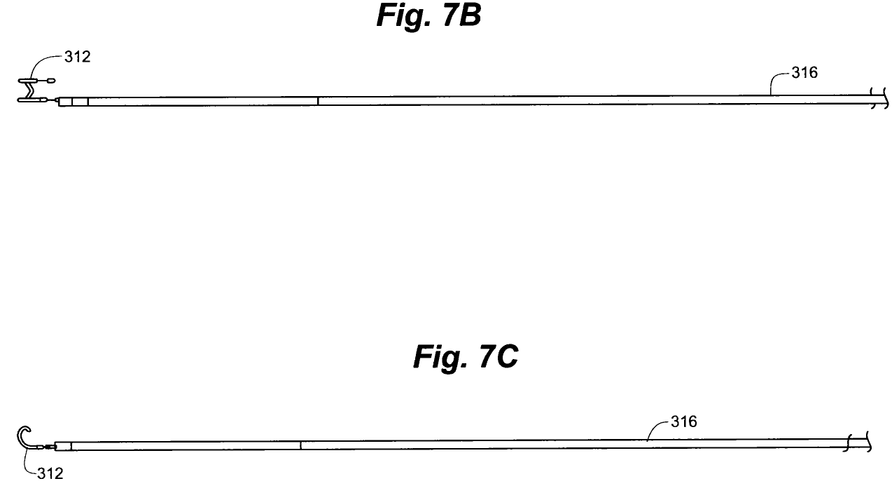

Embodiments of a delivery system 300 are depicted in FIGS. 6A-6E and 7A-7E. Delivery system 300 may be used for device implantation as well as for lead implantation. Delivery system 300 generally includes a device body 302 and a handle 304.

The body 302 can include a flexible wire 306. Wire 306 can be a coaxial wire that includes an inner wire 308 and an outer wire 310 that generally surrounds the inner wire 308. A grasper mechanism 312 can be disposed at a distal end of the inner wire 308. Body 302 can also include a flexible sheath 316 surrounding wire 306. The distal end of the flexible sheath 316 can include a collar section 314. The collar section 314 can have an inner diameter sized to incorporate not only the wire 306, but also the grasper mechanism 312. A secondary wire, or stylet, can also be contained in device body 302. Stylet can be contained within flexible sheath 316 in a separate lumen from wire 306, or can be contained in the same aperture as wire 306 and can be used to form or release a closed loop with grasper mechanism 312.

Handle 304 can define a central longitudinal aperture 318 that can accommodate body 302. A thumb slide 320 can be disposed within an axially positioned slot 322. Thumb slide 320 can be connected to a proximal end of wire 306. Handle 304 can also include a flush port 324. Flush port 324 can be used to flush blood out of delivery device 300 during implantation.

The grasper mechanism 312 has a collar or lasso like shape that permits selective frictional or pressure based grasping and releasing of the IID 200 or lead without the need for a positive mechanical mating of a mandrel as with prior device delivery solutions. The lasso, or lariat, includes a releasable loop 328, or honda, portion. The releasable honda 328 is configured to grasp the IID or a lead in order to position the IID or lead in the vasculature. Releasable honda 328 has an end portion 332 that can be connected to the collar section 314 or a stylet to form a closed loop.

The grasper mechanism 312 can have various shapes. The embodiment shown in FIGS. 6A-6E is generally hook shaped. Another embodiment of a grasper mechanism 312 suitable for device delivery depicted in FIGS. 7A-7E, is generally “w” shaped. Any shape that can be used with the collar section or a stylet to form a closed loop to grasp an IID can be used.

In one embodiment, grasper mechanism 312 can be formed of memory wire. The shape of the grasper mechanism 312 can be formed by setting the memory wire in a heated fixture. Memory wire allows the grasper mechanism to deform when necessary while still naturally retaining the grasping shape.

The thumb slide 320 can move within the axially disposed slot 322 in the handle 304. If the proximal end of inner wire 308 is connected to thumb slide 320, axial movement of the thumb slide 320 produces a corresponding movement of the inner wire 308. A stop fixture 330 can be disposed at the distal end of the slot 322, to prevent the thumb slide 320 from moving past a certain point. The stop fixture 330 reduces the potential for the inadvertent release of the IID 200 or lead during the implantation procedure. Alternatively, the thumb slide 320 can control the operation of a stylet contained within sheath 316 that is separate from wire 306. Alternatively, thumb slide 320 can be switched to operate an extension assembly coupled to outer wire 310, the extension assembly adapted to provide extension of the grasper mechanism.

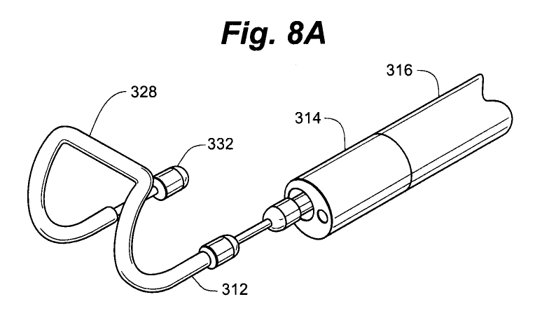

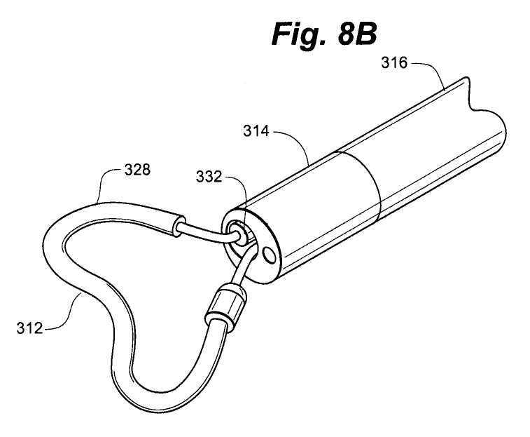

The grasper mechanism 312 can be configured to grasp and release the IID 200 or lead in various ways. In one embodiment, as can be seen in FIGS. 8A and 8B, the end portion 332 of releasable honda 328 of grasper mechanism 312 is manually inserted into collar section 314 of device body 302 to form a closed loop for grasping the IID 200 or lead. This can be done outside of the body on the operating table. To release the IID 200 or lead, the stop fixture 330 is removed allowing thumb slide 320 to advance into the region previously occupied by the stop fixture 330, pushing the inner wire 308 forward. As the inner wire 308 slides as far forward as it can go, the end portion 332 of releasable honda 328 is pulled out of collar section 314, thereby releasing the IID 200 or lead from the device delivery system's 300 grasp. In another embodiment, thumb slide 320 can control a stylet, which can be used to push end portion 332 out of collar section 314.

In another embodiment, a closed loop is formed around an IID 200 or lead by mating a stylet with the end portion 332 of releasable honda 328. To open the loop, the stop fixture 330 can be removed and the thumb slide 320 can move forward to push the inner wire 308 and end portion 332 out of contact with the stylet. Alternatively, the thumb slide 320 can control movement of the stylet and the loop can be opened by withdrawing the stylet out of contact with the end portion 332. In this alternative, it is possible to remotely grasp the IID 200 or lead while it is in or out of the body, by remotely moving the stylet forward with thumb slide 320 to close the loop with the end portion 332 around IID 200 or lead.

A distal end of device body 302 of delivery system can also be configured to articulate and/or rotate in order to aid in positioning of an IID 200 or lead 202. Articulation of the distal end of the delivery system can be accomplished through the use of one or more pull wires extending internally from the distal end of the device to the handle. The delivery system may be configured such that rotating the handle with respect to the flexible body pulls the internal wire, causing the articulation of the distal end of lead delivery system. In another embodiment, a supplemental thumb slide may be provided in handle, operably coupled to pull wire to cause articulation of the distal end.

As discussed above, IID 200 can be delivered using an over-the-wire technique. IID 200 can also be delivered without use of a guidewire. To implant IID 200 with delivery system 300, first an incision is formed to allow access to the vasculature. In one embodiment, the incision is formed in the femoral vein. An introducer sheath configured to allow insertion of devices into the vasculature while preventing loss of blood can be inserted into the incision to keep the vein open during the procedure.

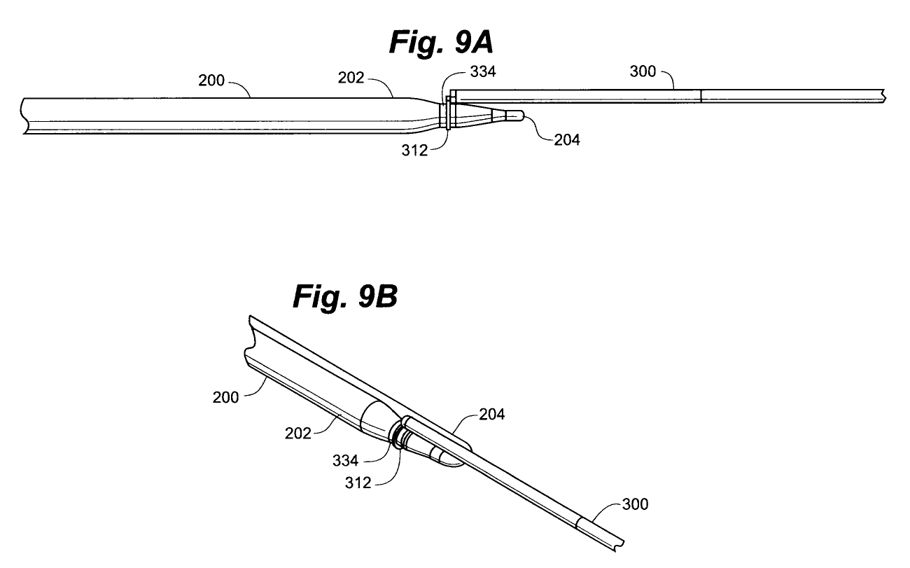

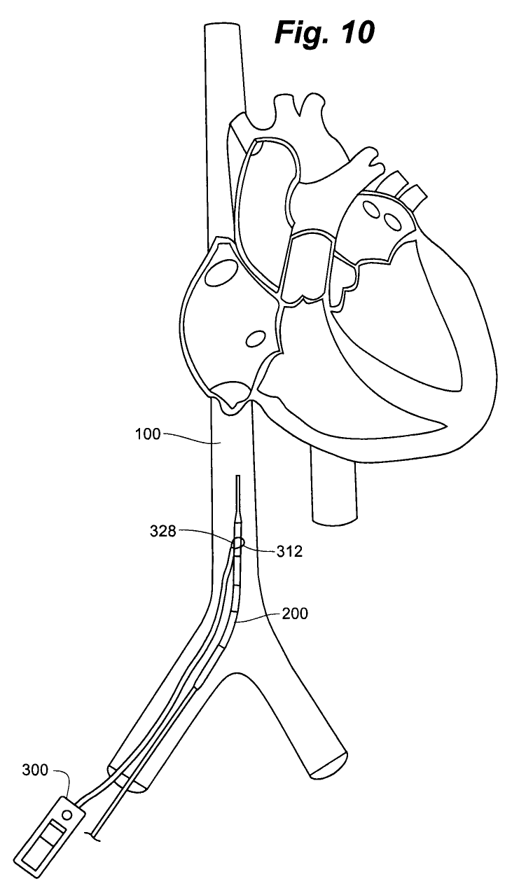

The delivery system 300 is then used to grasp the IID 200 with the grasper mechanism 312. As described above, the releasable honda 328 of device delivery system 300 can be manually secured around the IID 200 to grasp the IID 200 by various means prior to implantation. The grasper mechanism 312 can attach to the IID 200 at the proximal end, distal end, lead transition portion, or any other portion of the IID in order to position the IID at a desired location within the vasculature. In one embodiment depicted in FIGS. 9A and 9B, the delivery system 300 grasps the interface between the device body 204 and the lead 202. IID can be provided with a circumferential notch 334 configured to be grasped by the delivery system 300

The IID 200 can then be inserted through the introducer sheath. The delivery system 300 is used to guide the IID 200 through the vasculature to the desired location, as shown, for example, in FIG. 10. In one embodiment, the delivery system 300 maintains its grasp on the IID 200 until it reaches the desired location. In another embodiment, the delivery system 300 grasps a portion of the IID 200, for example its distal end 208, for a first part of the implantation process then releases the IID 200 and re-grasps it at a second location, for example its proximal end 206, to complete the implantation process. If at any point during the implantation process the delivery system 300 becomes overly filled with blood, flush port 324 on handle 304 can be used to flush the blood from the device 300. In some embodiments, fluoroscopy can be utilized to aid in all aspects of the implantation.

In some embodiments, once the IID 200 is positioned in its desired location, the delivery system 300 can release the IID 200 and be withdrawn from the body. In one embodiment, the IID 200 can be released by removing the lock 330 on handle 304 and operating the thumb slide 320 to open the loop with releasable honda 328. The thumb slide 320 can then be used to completely withdraw the grasper mechanism 312 into the delivery system 300 device body 302 to prevent it from contacting the patient's body as the delivery system 300 is removed. In another embodiment, delivery system 300 can retain position control until after a lead associated with the IID is implanted. Once the IID 200 has been implanted in the desired location and the delivery system 300 is withdrawn, the IID 200 can be anchored within the body. In one embodiment, the handle 304 remains completely outside of the patient's body during the entire implantation procedure.

### Anchor Delivery

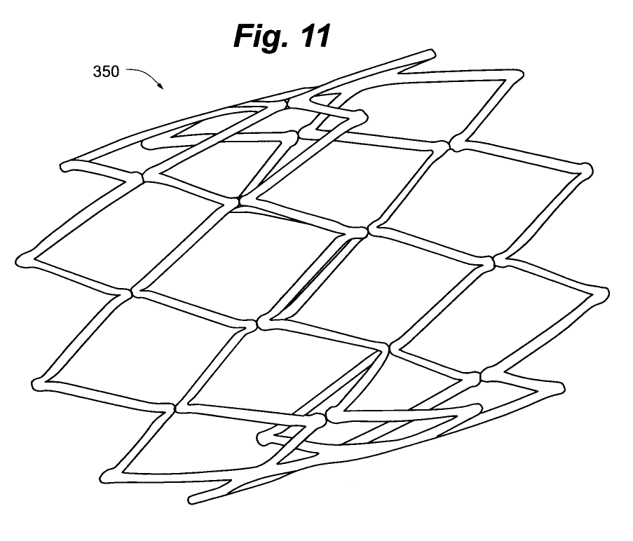

In one embodiment, an anchor delivery system includes a handle operably connected to a thumb switch or similar switch that activates an extension assembly to which an expandable anchor is positioned. In one embodiment, the anchor 350 comprises an intravascular stent, preferably of the type manufactured from a solid nitinol tube, having no welding points or filament crossings, such as depicted in FIG. 11. The stent has highly elastic properties and conforms smoothly to the anatomy. The anchor delivery system includes a coaxial sheath extending from the handle. The inner layer of the sheath may be a guidewire with an anchor loading zone at the distal end. The outer sheath substantially surrounds the inner sheath. The thumb switch is used to retract the outer sheath proximate the anchor when the anchor delivery system is disposed relative to the distal end of an IID within the vasculature.

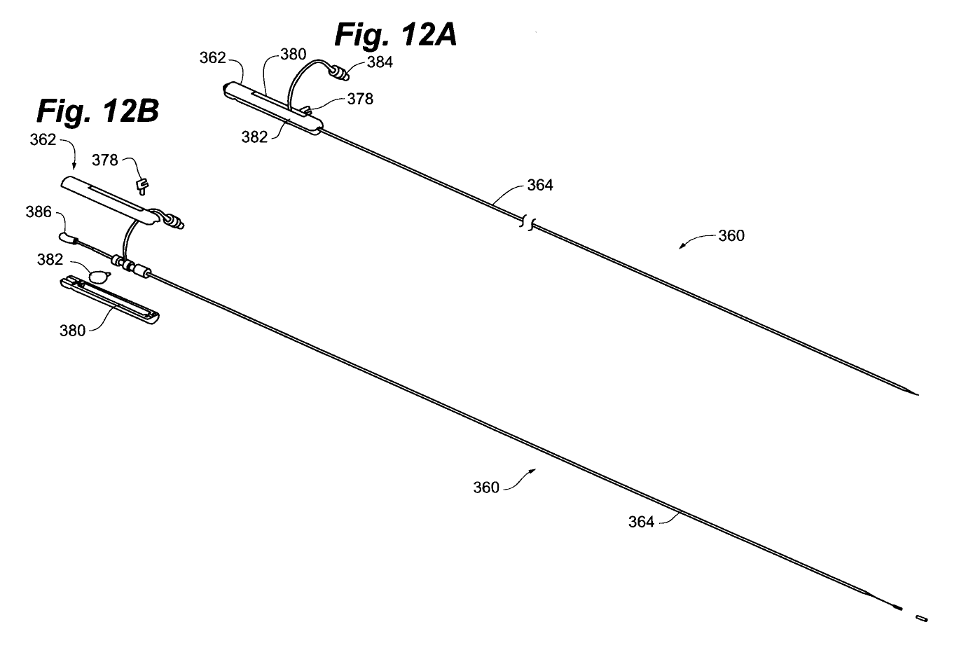

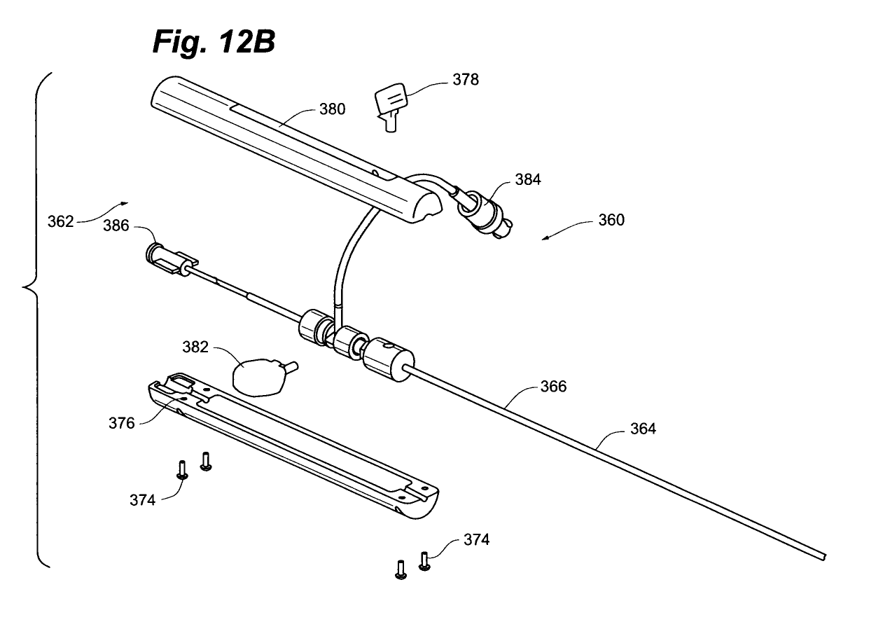

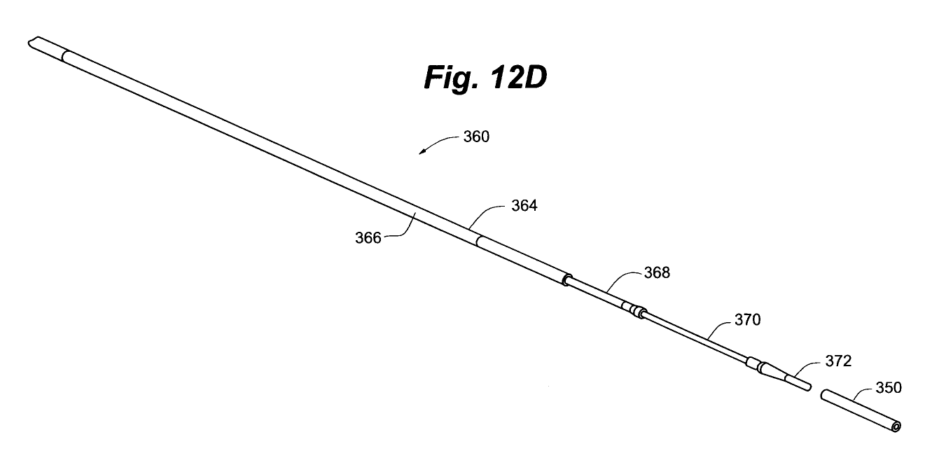

One embodiment of the anchor delivery system 360, depicted in FIGS. 12A-12D, provides a means for placing an anchor 350 proximate the anchor attachment of the IID. The anchor delivery system 360 includes a handle 362 supporting an anchor shaft assembly 364. Anchor shaft assembly 364 can include an anchor retention sheath 366 surrounding an inner shaft 368. An anchor holding area 370 can be positioned between a distal end of inner shaft 368 and a tip 372. The handle 362 may be a two-piece unit fastened together by connectors 374 inserted through connector apertures 376. A thumb switch 378 or retraction button is disposed within an axial slot 380 external to handle 362. The thumb switch 378 may be operably connected to anchor retention sheath 366 of the anchor shaft assembly 364. Operation of the thumb switch 378 causes anchor retention sheath 366 to retract relative to inner shaft 368. A safety pin 382 may be positioned at the distal end of slot 380 so as to prevent inadvertent anchor 350 release by movement of thumb switch 380. Removal of the safety pin 382 allows the thumb switch 380 to slide anchor retention sheath 366 back to expose anchor holding area 370. Alternatively, thumb switch 380 can be operably connected to inner shaft 368 and can expose anchor holding area 370 by moving inner shaft 368 forwardly relative to anchor retention sheath 366. Anchor delivery system 360 can also include a flush port 384 for flushing blood from between inner shaft 368 and anchor retention sheath 366 and flush port 386 for flushing blood from within inner shaft 368.

To deliver anchor 350 to a desired location in a patient's vasculature, an anchor 350 is threaded onto tip 372 and positioned in anchor holding area 370. Thumb switch 378 is then advanced forward to cover anchor 350 with anchor retention sheath 366, thereby compressing anchor 350 and holding it in place in anchor holding area 370. Safety pin 382 can be inserted in slot 380 to prevent inadvertent release of anchor 350 during implantation. Anchor delivery system 360 is then used to guide anchor 350 to a desired location in the patient's vasculature. Safety pin 382 can be removed and thumb switch 378 can be used to slide back anchor retention sheath 366 to release anchor 350. The tip 372 can then be withdrawn through the expanded anchor 350, leaving the anchor 350 situated at the desired location, and the anchor delivery system 360 can be withdrawn from the body.

In one embodiment, the handle 362 can define an axial central aperture in which the anchor shaft assembly 364 is operably connected to a connector with a side arm to which a polyvinyl tube extends. The connector can be used for introducing and aspirating fluids through the extension shaft assembly. Connector is fluidly connected to a valve and thumb control seat.

In another embodiment, anchor can be integrated with the IID. One example of an anchor integrated with an IID is disclosed in U.S. Published Appl. No. 2005/0228471. Such an anchor would therefore be implanted along with the IID, as described above.

### Lead Delivery

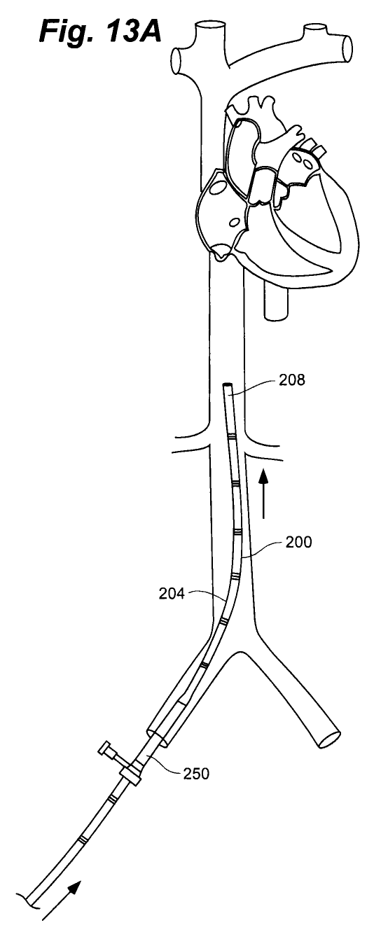

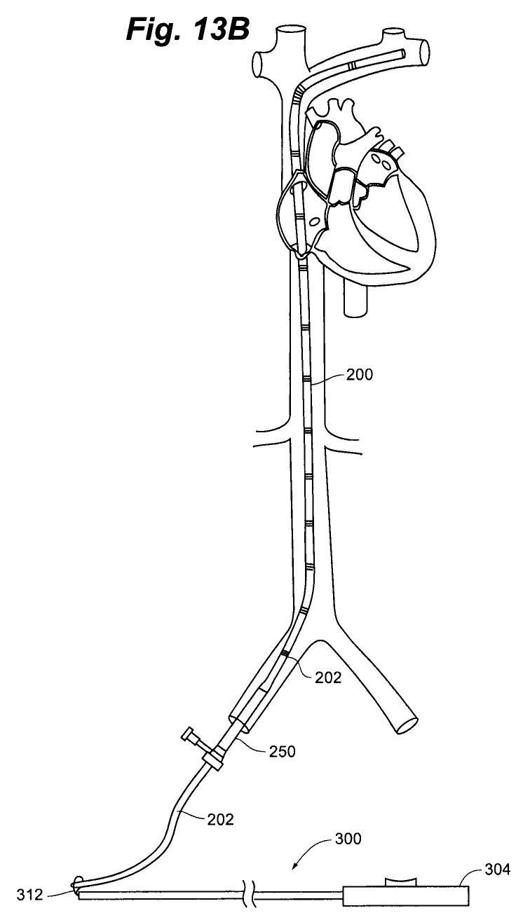

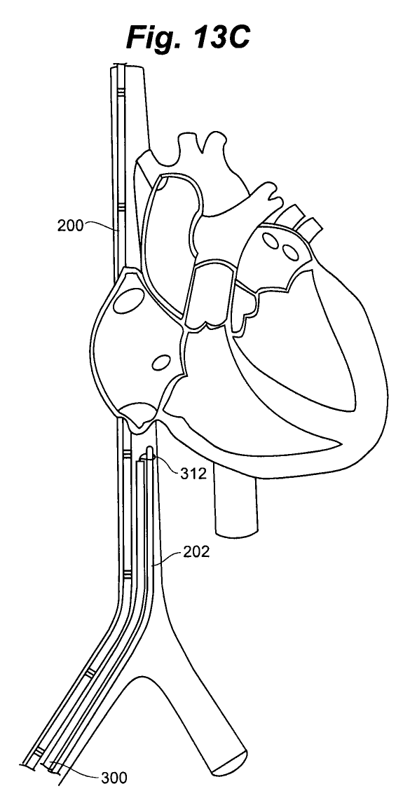

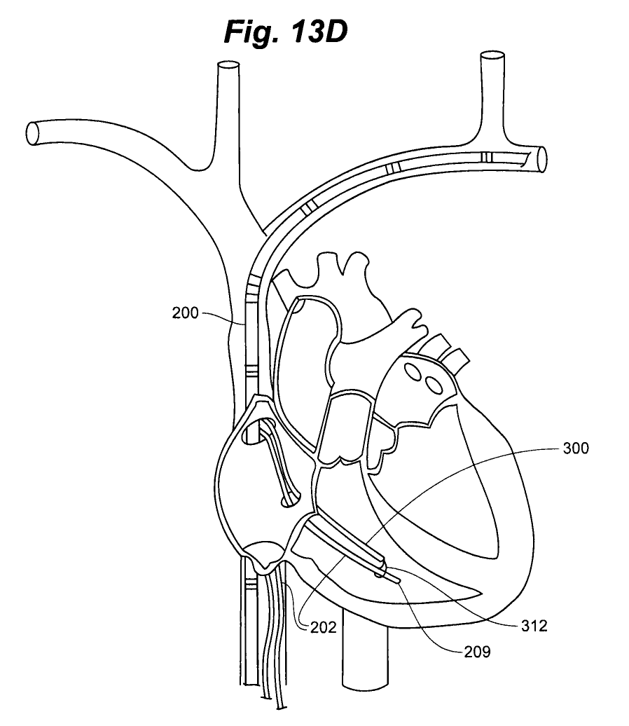

Grasper-style lead delivery tools are generally of identical structure and functionality to delivery tool 300 described above. Generally, but not always, following device delivery, delivery tool 300 can be used to deliver lead 202 to a desired location. Referring now to FIGS. 13A-13D, one embodiment of lead implantation is depicted wherein implantation of lead 202 occurs subsequent to the delivery and placement of the IID 200 to which lead 202 is attached. IID 200 is positioned such that the proximal end of lead 202, being integrally formed with the proximal end 206 of IID 200, is located generally within or near the inferior vena cava. Utilizing the grasper-style lead delivery system 300, the distal end of lead 202 is securely grasped by releasable honda 328. Distal end of lead 202 may be situated within the vasculature, or may be extending through the introducer sheath and residing at least partly outside of the patient, as depicted in FIG. 13B. Lead 202 may be grasped at or near its distal end, to provide positive directional control of the distal end of the lead during implantation. The precise grasping location on distal end of lead 202 will be dependent on the particular structure of lead 202, such as the location of any electrodes or fixation elements.

Once lead 202 is securely grasped, lead delivery system 300 is used to navigate lead 202, distal end first, through the inferior vena cava toward the heart, as depicted in FIG. 13C. To guide lead 202 into the heart from the inferior vena cava, lead delivery system 300 may be rotated, articulated, extended or any combination thereof so as to navigate the acute angle from the inferior vena cava into the right atrium and avoid damage or interference with tissue in and around the heart. Additional manipulation of lead delivery system 300 may be required to guide lead 202 to its desired location within the heart, as depicted in FIG. 13D.

In another embodiment of lead implantation, lead 202 may be partially delivered to its desired location with lead delivery system 300, released from delivery system 300, and re-grasped at another portion of lead 202. Such method permits grasping lead 202 at a desired position during a first phase of implantation, and at a second position during a second phase of implantation.

In a further embodiment, lead delivery system 300 is provided with an internal lumen configured for receipt of a guidewire or steerable stylet. A pre-shaped guidewire may be first implanted with a guidewire introducer catheter, the pre-shaped guidewire defining a pathway from the desired implant location for lead 202 within the heart, through the inferior vena cava, and out the introducer sheath positioned in the femoral vein. After withdrawing the guidewire introducer catheter, lead 202 is grasped with releasable honda 328 and lead delivery system 300 is advanced onto the guidewire. Lead delivery system 300 with lead 202 releasably attached is introduced along the guidewire to the desired implant location. During implantation, if needed the guidewire can be removed from the lumen in lead delivery system 300, reshaped, and then reinserted into the lumen in lead delivery system 300 so as to alter the path along which lead delivery system 300 and lead 202 are advanced.

In another embodiment wherein lead delivery system 300 includes an internal lumen, a steerable stylet is provided for assisting in delivery of lead 202. Releasable honda 328 is used to grasp lead 202, and lead delivery system 300 and lead 202 are positioned within the inferior vena cava. The steerable stylet is introduced in the proximal end of lead delivery system body 204. As lead delivery system 300, with lead 202 attached, is advanced through the vasculature, steerable stylet may be used to control, or supplement the control of, the direction of lead delivery system 300.

Upon successful delivery of lead 202 to its desired location, in one embodiment lead 202 is released from lead delivery system 300 to allow for passive fixation of lead 202. In another embodiment, lead delivery system 300 is withdrawn prior to fixation of lead 202, so as to allow introduction of a fixation device for securing the lead in place. Withdrawal of lead delivery system 300 includes releasing honda 328 from lead 202, such as by releasing stop fixture 330 and operating thumb slide 320 to open the lasso. Thumb slide can further be operated to completely withdraw grasper mechanism 312 into the device delivery system 300 device body 302, to prevent grasper mechanism 312 from contacting vessel walls during withdrawal.

In another embodiment wherein lead delivery system 300 includes a lumen for a stylet, a stylet is introduced through the lumen to the lasso, and used to disengage end portion 332 of releasable honda 328 from collar section 314 of device body 302.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

1. A system for implanting an intravascular implantable device, comprising:
an intravascular implantable electrophysiological device having an elongate device body and a cardiac lead coupled to the device body that are adapted to be implanted within the vasculature of a patient, the device body including circuitry adapted to deliver electrophysiological therapy through the cardiac lead, the cardiac lead having a distal end including an electrode and being adapted for fixation within a heart of the patient and a proximal end non-releasably coupled to a proximal end of the device body;
a device delivery tool having an elongated flexible body, a handle operably coupled to a proximal end of the flexible body, and a grasper mechanism including a releasable honda operably coupled to a distal end of the flexible device body, wherein the device delivery tool is adapted to releasably grasp and deliver the intravascular implantable device body; and
a lead delivery tool adapted to deliver the cardiac lead through the vasculature and to a desired implant location within the heart.

2. The system of claim 1, wherein the lead delivery tool comprises the device delivery tool.

3. The system of claim 1, further comprising an anchor delivery tool having an elongated flexible body and a handle operably coupled to a proximal end of the flexible body, wherein the flexible body includes an anchor holding portion at a distal end of the flexible body, the anchor holding portion selectively retractable with operation of the handle to release a compressed anchor from within anchor holding portion.

4. The system of claim 1, further comprising an introducer sheath having a hemostasis valve and an introducer shaft and a central aperture defined along a longitudinal axis of the introducer sheath, the hemostasis valve including a selectively compressible seal configured to seal at least a part of the central aperture.

5. The system of claim 1, further comprising a guidewire configured to be temporarily introduced within the vasculature of the patient and define a delivery path over which the intravascular implantable device may be routed, and wherein the intravascular implantable device includes a tip portion on the distal end of the device body, the tip portion having a guidewire passage adapted to receive a guidewire therein.

6. A method of implanting an intravascular implantable electrophysiological device having a device body and a cardiac lead coupled to the device body, comprising:
providing an intravascular implantable electrophysiological device having an elongate device body and a cardiac lead coupled to the device body that are adapted to be implanted within the vasculature of a patient, the device body including circuitry adapted to deliver electrophysiological therapy through the cardiac lead, the cardiac lead having a distal end including an electrode and being adapted for fixation within a heart of the patient and a proximal end non-releasably coupled to a proximal end of the device body;
providing a device delivery tool having an elongated flexible body, a handle operably coupled to a proximal end of the flexible body, and a grasper mechanism including a releasable honda operably coupled to a distal end of the flexible device body, wherein the device delivery tool is adapted to releasably grasp and deliver the intravascular implantable device body;
providing a lead delivery tool adapted to deliver the cardiac lead through the vasculature and to a desired implant location within the heart; and
providing instructions, including:
obtaining access to a portion of the vasculature of a patient;
grasping the intravascular implantable device with the device delivery tool by closing a releasable honda around the device;
inserting the intravascular implantable device into the patient's vasculature;
guiding the intravascular implantable device to a desired location within the patient's body using the device delivery tool;
with at least a portion of the lead external to the patient, releasably securing the lead with the lead delivery tool;
delivering the distal end of the cardiac lead through the vasculature with the lead delivery tool to a desired location within the heart of the patient;
fixating the distal end of the cardiac lead at the desired location; and
releasing the cardiac lead from the lead delivery tool and removing the lead delivery tool from the patient.

7. The method of 6, wherein the device delivery tool and the lead delivery tool comprise the same tool and wherein releasably securing the lead with the lead delivery tool comprises releasably grasping an exterior of the lead proximate the distal end of the lead with the grasper mechanism of the lead delivery tool.

8. The method of 6, further comprising:
providing an anchor delivery tool configured to deliver a self-expanding anchor to a desired location in a compressed position and release the anchor from the anchor delivery tool such that the anchor expands to a deployed position, anchoring the implantable intravascular device within the vasculature.

9. The method of 6 further comprising providing a guidewire, wherein the intravascular implantable device includes a tip portion on the distal end of the device body, the tip portion including a guidewire passage, and the instructions further include:
delivering a guidewire through the vasculature of the patient to a desired implant location prior to inserting the intravascular implantable device into the patient's vasculature;
inserting the guidewire into the guidewire passage and inserting the intravascular implantable device into the patient's vasculature; and
guiding the intravascular implantable device along the guidewire to a desired location within the patient's body using the delivery tool.

10. The method of 6, wherein the instructions further comprise:
grasping the proximal end of the intravascular implantable device with the grasper mechanism by closing a releasable honda around the device; and
releasably grasping an exterior of the lead proximate the distal end of the lead with a grasper mechanism of a second delivery tool.

11. The method of 6, wherein delivering the distal end of the cardiac lead through the vasculature further comprises delivering the cardiac lead through an inferior cava of the patient and into a right ventricle of the heart of the patient.

12. An apparatus for positioning an intravascular implantable device in a patient's body, comprising:
an elongated flexible device body adapted to be temporarily introduced into the patient's vasculature, the device body having a proximal end and a distal end and including a flexible wire extending through at least a portion of the length of the device body;
a handle operably connected to the proximal end of the device body; and
a grasper mechanism connected to the distal end of the flexible wire and configured to releasably grasp an intravascular implantable device having an elongated cylindrical housing, wherein the grasper mechanism is configured to grasp the intravascular implantable device by closing a releasable honda around the cylindrical housing of the device that is selectively controllable with the handle.

13. The apparatus of claim 12, wherein the releasable honda is closed around the intravascular implantable device by interfacing an end portion of the releasable honda with a collar section located at the distal end of the device body.

14. The apparatus of claim 12, wherein the device body further includes a stylet extending through at least a portion of the length of the device body, and wherein the releasable honda is closed around the intravascular implantable device by connecting the stylet with the releasable honda.

15. The apparatus of claim 12, wherein the grasper mechanism is configured to release the intravascular implantable device via operation of the handle.

16. The apparatus of claim 12, wherein the handle includes a slide connected to a proximal end of the flexible wire, and wherein axial movement of the slide relative to the handle produces a corresponding movement of the flexible wire.

17. The apparatus of claim 16, wherein the handle further includes a stop fixture configured to prevent movement of the slide beyond a predetermined position on the handle, and wherein, in response to release of the stop fixture, movement of the handle beyond the predetermined position causes the releasable honda to release its grasp on the intravascular implantable device.

18. The apparatus of claim 12, wherein the device body further includes a flexible sheath surrounding the wire.

19. The apparatus of claim 12, wherein the grasper mechanism can be completely withdrawn into the device body via operation of the handle.
